(19) United States
(12) Patent Application Publication
(10) Pub. No.: US 2010/0018607 A1
(43) Pub. Date: Jan. 28, 2010

(54) BOTTLE VALVE WITH BIASING MEMBER

(76) Inventor: Michael CUZYDLO, Orchard Park, NY (US)

Correspondence Address: REED SMITH LLP, P.O. BOX 488, PITTSBURGH, PA, 15230-0488, US

(21) Appl. No.: 12/507,478

(22) Filed: Jul. 22, 2009

Related U.S. Application Data
(60) Provisional application No. 61/082,624, filed on Jul. 22, 2008.

(51) Int. Cl. A61M 16/10; B67D 7/84

(52) U.S. Cl. 141/311 R; 222/173

(57) ABSTRACT

An apparatus and system for transferring a liquid, such as a liquid anesthetic, from a reservoir to a machine while minimizing the release of the liquid to the surrounding environment. The apparatus can include a frame member defining a frame passage extending from a first end of the frame member to a second end of the frame member and a nozzle connected to the frame member. The nozzle can be moveable between a first position and a second position relative to the frame member, and include a biasing member connected to the frame member and the nozzle.

### CROSS-REFERENCE TO RELATED APPLICATION

This application claims the benefit under 35 U.S.C. § 119(e) of the earlier filing date of U.S. Provisional Application Ser. No. 61/082,624 filed on Jul. 22, 2008, the entire disclosure of which is hereby incorporated by reference herein as if being set forth in its entirety.

### FIELD OF THE INVENTION

This application discloses an invention which is related, generally and in various embodiments, to a device for supplying a liquid to a machine, such as a vaporizer.

### BACKGROUND OF THE INVENTION

Liquid anesthetics are often packaged in glass bottles and shipped to a location where they may be used to anesthetize a patient undergoing a medical or dental procedure. Such anesthetics may also be used to induce analgesia in a patient undergoing a medical or dental procedure. In order to administer the anesthetic, the contents of the glass bottle are placed in a vaporizer. The vaporizer can vaporize the anesthetic and provide the vaporized anesthetic in a desired amount to the patient.

Inhalable anesthetics are typically volatile substances with relatively low boiling points and high vapor pressure. Preferably, there is little or no release of anesthetic to the atmosphere during handing. To transfer liquid anesthetic to a vaporizer, however, the bottle containing the vaporizer must be opened. Since it is unwise to expose medical personnel performing a procedure to an anesthetic, and since anesthetics are expensive, devices have been developed to minimize the release of anesthetic from a bottle to the environment surrounding a vaporizer. These devices, however, have failed to effectively minimize the release of anesthetic.

### SUMMARY OF THE INVENTION

This application discloses an apparatus and system for transferring a liquid, such as an anesthetic, from a reservoir to a machine while effectively minimizing the release of the liquid to the surrounding environment. The apparatus can include a frame member defining a frame passage extending from a first end of the frame member to a second end of the frame member and a nozzle connected to the frame member. The nozzle can be moveable between a first position and a second position relative to the frame member, and can define a plug member configured to block the second end of the frame passage when the nozzle is in the first position. The apparatus can further include a biasing member connected to the frame member and the nozzle.

### BRIEF DESCRIPTION OF THE DRAWINGS

FIG. 1 illustrates a side view of a reservoir and a closing mechanism for connecting the reservoir to a vaporizer.

FIG. 2A illustrates a vaporizer receiving station and closing mechanism according to an alignable opening embodiment of the invention in a closed position, and FIG. 2B is a cross-sectional view taken along line A-A in FIG. 2A.

FIG. 3A illustrates a vaporizer receiving station and a closing mechanism according to an alignable opening embodiment of the invention in an open position, and FIG. 3B is a cross-sectional view taken along line A-A in FIG. 3A.

FIG. 4A illustrates a vaporizer receiving station and a closing mechanism according to a sealing disk embodiment of the invention in a closed position, and FIG. 4B is a cross-sectional view taken along line A-A in FIG. 4A.

FIG. 5A illustrates a vaporizer receiving station and a closing mechanism according to a sealing disk embodiment of the invention in an open position, and FIG. 5B is a cross-sectional view taken along line A-A in FIG. 5A.

### DETAILED DESCRIPTION OF THE INVENTION

The accompanying drawings are intended to provide further understanding of the invention and are incorporated in and constitute a part of the description of the invention. The drawings illustrate an embodiment of the invention and together with the description illustrate principles of the invention.

The drawings should not be taken as implying any necessary limitation on the essential scope of invention. The drawings are given by way of non-limitative example to explain the nature of the invention.

For a more complete understanding of the instant invention reference is now made to the following description taken in conjunction with accompanying drawings.

The various features of novelty which characterize the invention are pointed out specifically in the claims which are a part of this description. For a better understanding of the invention, reference should be made to the drawings and descriptive matter in which there are illustrated and described preferred embodiments of invention.

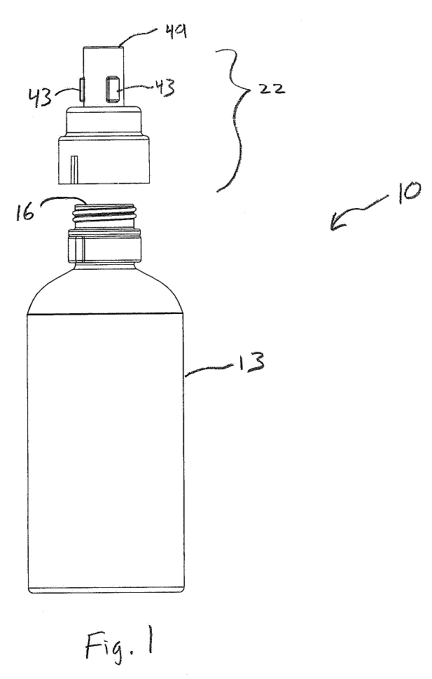

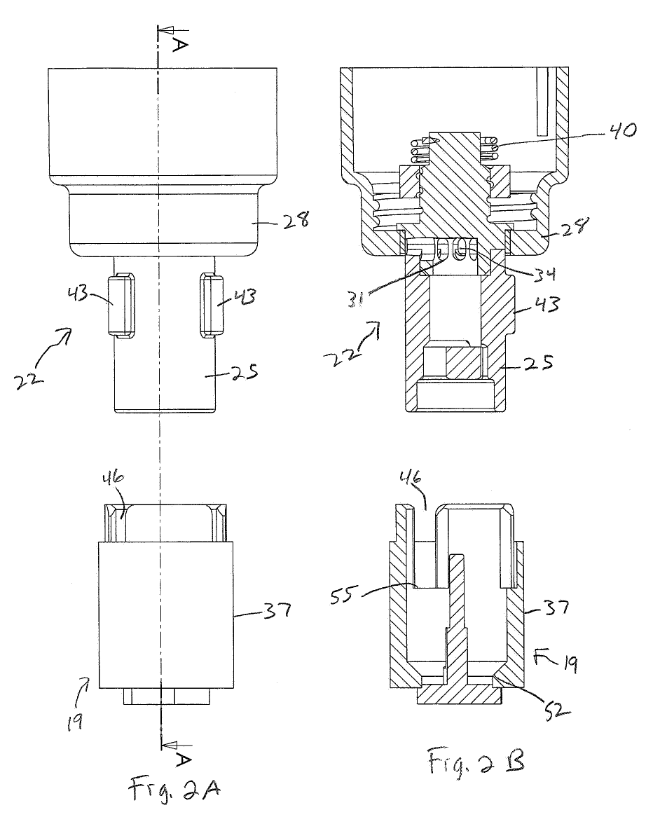

Referring now to the drawings, wherein like numerals designate identical or corresponding parts throughout the referred views, FIG. 1 shows an embodiment of an apparatus 10 which may hold a liquid such as a volatile anesthetic. The apparatus 10 may include a reservoir 13 which can be a glass bottle or any other container capable of containing a liquid. At its top, the reservoir 13 includes an opening 16 through which liquid anesthetic may be transferred from the reservoir 13 to the apparatus 22, and ultimately to the machine 19. The apparatus may include a closing mechanism 22 connecting a reservoir 13 to a machine 19 (FIGS. 2A-5B). In this embodiment, the machine 19 is a vaporizer that dispenses anesthetic to a person undergoing a medical procedure and the apparatus 22 is a closing mechanism connecting the vaporizer 19 the reservoir 13 for selectively allowing the volatile anesthetic to leave the reservoir.

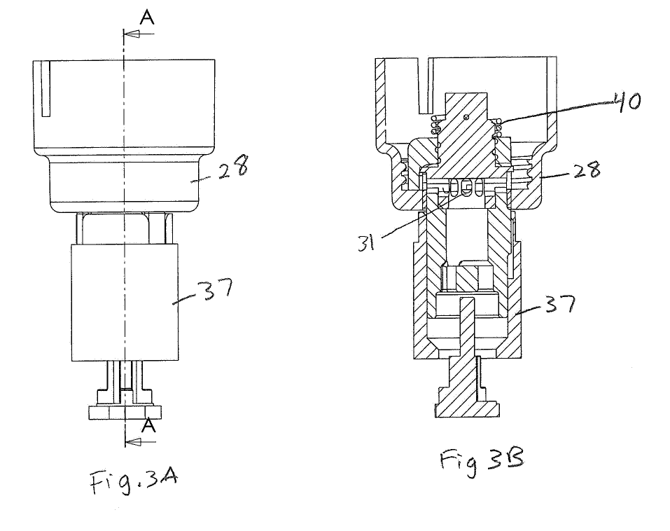

FIGS. 2A-3B provide views of a closing mechanism 22 according to an alignable opening embodiment of the invention for selectively allowing liquid to transfer from a reservoir 13 to a vaporizer 19. The closing mechanism 22 includes a frame member 28 and a nozzle 25 which are moveable relative to each other. In embodiments of the invention, a portion of the nozzle 25 overlaps with and forms a seal with a portion of the frame 28. The nozzle and the frame 28 may each include openings 31, 34. The nozzle 25 is connected to the frame member 28, and the nozzle is moveable between a first position (FIGS. 2A-2B) and a second position (FIGS. 3A-3B) relative to the frame member 28. In the first position (FIGS. 2A-2B) of the nozzle in which the openings 31 in the nozzle 25 are aligned with the openings 34 in the frame 28, the passage of liquid within the closing mechanism 22 is prevented. In the second position (FIGS. 3A-3B) in which the nozzle openings 31 are not aligned with the frame openings 34 passage of liquid within the closing mechanism 22 is allowed.

In this embodiment, the frame member 28 may be screwed to the reservoir 13 and held to the reservoir via an adhesive or by connected any other commonly known method such as methods allowing removal of the frame 28 and nozzle 25 from the reservoir 13 such as, for example, a threaded surface, a self-locking snap ring, or a crimped ring. In this manner, the closing mechanism 22 and reservoir 13 may be cleaned and reused.

A biasing member 40 can be connected at one end to a base 12 of the frame member 28 and at the opposite end to the nozzle 25. The biasing member 40 can be a spring, such as a torsion spring. As will be discussed in greater detail below, the biasing member 40 can apply a biasing force between the frame member 28 and the nozzle 25 so that the nozzle openings 31 are not aligned with the frame openings 34 to prevent the flow of a liquid. In this manner the anesthetic can not escape unless a force is applied to counter the force imposed by the torsion spring 40.

The nozzle 25 may include activation extensions 43. The nozzle 25 and extensions may fit into channels 46 provided by the receiving stations 37 of the vaporizer 19. To insert the nozzle 25 into the receiving station 37, the activation extensions 43 are aligned with the openings 31, 34, and the reservoir 13 is pushed toward the receiving station 37. Once the nozzle 25 is pushed by the reservoir 13 into the receiving station 37, the reservoir 13, including the attached frame 28, may be rotated while the receiving station 37 prevents the nozzle 25 from rotating to the same extent as the frame 28. In doing so, the frame openings 34 are allowed to align with the nozzle openings 31, thereby allowing anesthetic to leave the reservoir 13 through the nozzle 25 and enter the receiving station 37. By sizing the channels 46 properly, one or more sealing surfaces 49 of the nozzle 25 may be brought into contact with one or more sealing surfaces 52 of the receiving station 37 before the openings 31, 34 are aligned to allow anesthetic to leave the reservoir 13.

Channels 46 in the receiving station 37 may be sized to accept the activation extensions 43 as the nozzle 25 is inserted into the receiving station 37. When the nozzle 25 has been inserted to a desired extent, the channels 46 may permit the activation extensions 43 to rotate slightly as the reservoir 13 is rotated, so that each activation extension 43 contacts an abutment 55 at the end of each channel 46. The abutment prevents the activation extensions 43 from further rotating as the reservoir 13 continues to rotate, thereby aligning the opening 31, 34 only after the activation extensions 43 are against the abutment 55. If the activation extensions 43 are allowed to rotate slightly in the channels 46 before contacting the abutments 55, the nozzle 25 may be held within the receiving station 37 until the nozzle 25 is rotated in the opposite direction. When the nozzle 25 is held within the receiving station 37, the device is prevented from being removed from the receiving station while the openings 31, 34 are aligned. The channels 46 may be any suitable shape. In embodiments of the invention, each channel 46 is L-shaped.

Once a desired amount of the anesthetic has been emptied from the reservoir 13 to the vaporizer 19, the reservoir 13 may be rotated in the opposite direction from that which aligned the openings 31, 34, and while the torsion spring 40 applies a force, the activation extensions 43 will be held against the abutments 55. Ultimately, the nozzle openings 31 will come out of alignment with the frame openings 34 as the reservoir 13 is rotated in that opposite direction. At about the same time the frame openings 34 are in the non-aligned position, the torsions spring 40 will no longer exert a force to hold the activation extensions 43 against the abutments 55, and the nozzle 25 may be allowed to rotate to the same extent as reservoir 13, and the activation extensions 43 may be removed from the channels 46.

In this fashion, the closing mechanism 22 will prevent anesthetic from leaving the reservoir 13 unless the activation extension 43 are engaged with the receiving station 37. By doing so, medical personnel will be protected from exposure to the anesthetic while the anesthetic is transferred to the vaporizer 19.

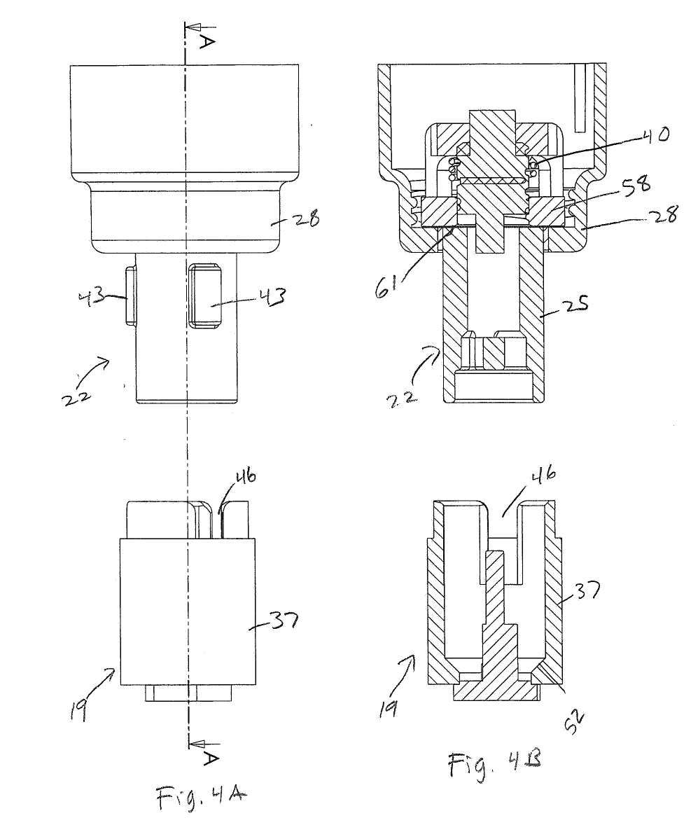

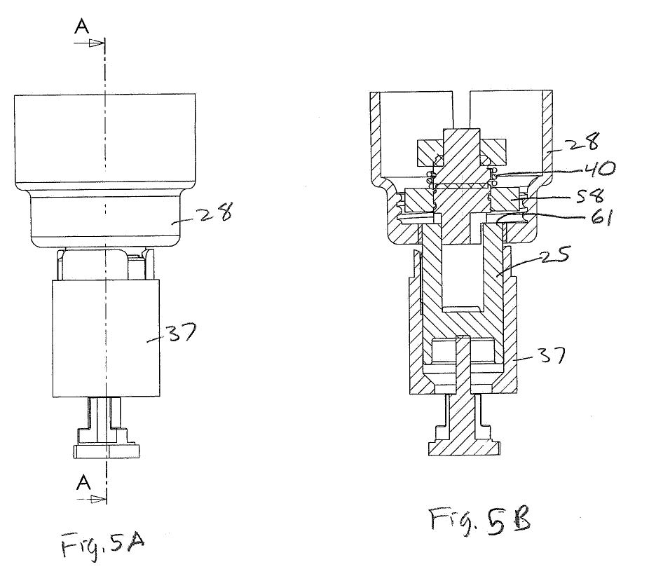

In other various embodiments of the device including the sealing disk embodiment shown in FIGS. 4A-5B, a sealing disk 58 is provide in lieu of nozzle openings 31 that align with frame openings 34. As in the first embodiment, the nozzle is moveable between a first position (FIGS. 4A-4B) and a second position (FIGS. 5A-5B) relative to the frame member 28. In the first position (FIGS. 4A-4B) of the nozzle the torsion spring 40 biases the relative positions of the frame 28 and nozzle 25 to keep the sealing disk 58 against a sealing surface 61 when no forces counter the spring force, the passage of liquid within the closing mechanism 22 is prevented. In the second position (FIGS. 5A-5B), rotation of the frame 28 relative to the nozzle 25 causes the sealing disk 58 to move away from the sealing surface 61 in order to allow anesthetic to leave the reservoir 13 via the nozzle 25.

It is to be understood that the descriptions of the invention have been simplified to illustrate characteristics that are relevant for a clear understanding of the invention. Those of ordinary skill in the art may recognize that other elements or steps are desirable or required in implementing the invention. However, because such elements or steps are well known in the art, and because they do not facilitate a better understanding of the invention, a discussion of such elements or steps is not provided herein. The disclosure herein is directed to all such variations and modifications to such elements and methods known to those skilled in the art.

It is to be understood that the invention may assume various alternative orientations and step sequences, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

What is claimed is:

1. An apparatus comprising:
a frame member, wherein the frame member defines a frame passage extending from a first end of the frame member to a second end of the frame member;
a nozzle connected to the frame member, wherein (a) the nozzle is moveable between a first position and a second position relative to the frame member; and
a biasing member connected to the frame member and the nozzle.

2. The apparatus of claim 1, wherein the frame member comprises a base configured for connection to a reservoir containing a liquid.

3. The apparatus of claim 2, wherein the base of the frame member is configured for connection to the reservoir by adhesive.

4. The apparatus of claim 1, wherein the nozzle surrounds the frame member.

5. The apparatus of claim 1, wherein the nozzle is threadedly connected to the frame member.

6. The apparatus of claim 1, wherein the frame member defines a threading configured for engagement with the nozzle.

7. The apparatus of claim 1, wherein the nozzle defines a threading configured for engagement with the frame member.

8. The apparatus of claim 1, wherein the nozzle is configured for connection to a machine.

9. The apparatus of claim 8, wherein the nozzle defines a threading for the connection to the machine.

10. The apparatus of claim 8, wherein the machine is a vaporizer.

11. The apparatus of claim 1, wherein the nozzle includes radially extending openings and the frame includes radially extending openings which, when in the first position, create a pathway through the nozzle when the openings are aligned, and, when in the second position, prevent a pathway through the nozzle when the openings re not aligned.

12. The apparatus of claim 1, wherein the nozzle includes a sealing surface and the frame includes a sealing disk which, when in the first position, create a pathway through the nozzle when the sealing disk is not in contact with the sealing surface, and, when in the second position, prevent a pathway through the nozzle when the sealing disk is in contact with the sealing surface.

13. The apparatus of claim 1, wherein the nozzle includes activation extensions adapted for being receivable in corresponding channels in a receiving station of a vaporizer.

14. The apparatus of claim 1, wherein the biasing member is a spring.

15. The apparatus of claim 1, wherein the biasing member applies a biasing force between the frame and the nozzle so that the nozzle is normally in the first position.

16. A system, comprising:
a reservoir containing a liquid; and
an apparatus connected to the reservoir, wherein the apparatus comprises:
a frame member, wherein the frame member defines a frame passage extending from a first end of the frame member to a second end of the frame member;
a nozzle connected to the frame member, wherein the nozzle (a) is moveable between a first position and a second position relative to the frame member; and
a biasing member connected to the frame member and the nozzle.

17. The system of claim 16, wherein the nozzle includes radially extending openings and the frame includes radially extending openings which, when in the first position, create a pathway through the nozzle when the openings are aligned, and, when in the second position, prevent a pathway through the nozzle when the openings re not aligned.

18. The system of claim 16, wherein the nozzle includes a sealing surface and the frame includes a sealing disk which, when in the first position, create a pathway through the nozzle when the sealing disk is not in contact with the sealing surface, and, when in the second position, prevent a pathway through the nozzle when the sealing disk is in contact with the sealing surface.

19. The system of claim 16, wherein the nozzle includes activation extensions adapted for being receivable in corresponding channels in a receiving station of a vaporizer.

20. The system of claim 16, further comprising a machine connected to the apparatus.

21. The system of claim 20, wherein the machine is a vaporizer.
